(19) United States
(12) Patent Application Publication
(10) Pub. No.: US 2018/0110417 A1
(43) Pub. Date: Apr. 26, 2018

(54) FIBER OPTIC TEMPERATURE MEASUREMENT SYSTEM

(71) Applicant: The University of Massachusetts, Boston, MA (US)

(72) Inventors: Xingwei Wang, Shrewsbury, MA (US); Nan Wu, Sunderland, MA (US)

(21) Appl. No.: 15/569,113

(22) PCT Filed: Jun. 3, 2016
(86) PCT No.: PCT/US16/35748

§ 371 Date: Oct. 25, 2017

Related U.S. Application Data
(60) Provisional application No. 62/170,764, filed on Jun. 4, 2015.

(51) Int. Cl. A61B 5/01; A61B 8/12; G01H 9/00; A61B 5/00; G01S 15/89; A61B 8/00

(52) U.S. Cl. CPC A61B 5/015; A61B 8/12; G01H 9/004; A61B 5/6852; G01S 15/8965; A61B 8/4483; A61B 5/4836

(57) ABSTRACT

In an embodiment, a transducer includes a first optical fiber having an ultrasound generator including a nanoparticulate material on an end thereof; and a second optical fiber having an ultrasound detector on an end thereof, wherein the ultrasound detector includes a Fabry Perot cavity. In another embodiment, a transducer includes an ultrasound generator comprising a nanoparticulate material and an ultrasound detector comprising a Fabry Perot cavity, wherein both the ultrasound generator and the ultrasound detector are disposed on an end of a first optical fiber.

### CROSS-REFERENCE TO RELATED APPLICATION

This application claims the benefit of U.S. Provisional Patent Application Ser. No. 62/170,764 filed Jun. 4, 2015. The related application is incorporated herein in its entirety by reference.

### BACKGROUND

### (1) Field

This disclosure relates to a fiber optic temperature measurement system, and a method of determining a treatment endpoint using the fiber optic temperature measurement system.

### (2) Description of the Related Art

Ablation is an invasive technique that is used in the treatment of atrial fibrillation (Afib), a common cardiac arrhythmia. Atrial fibrillation is a heart rhythm disorder characterized by rapid, irregular, and chaotic electrical activity in the atria. Ablation involves the removal of an unwanted structure or tissue. A type of ablation is catheter ablation. In catheter ablation, a catheter is inserted into a blood vessel, typically in the groin, and guided through the blood vessels into the heart. When the tip of the catheter is placed against the part of the heart causing the arrhythmia, radiofrequency electrical current is applied through the catheter to produce a small burn about 6 to 8 mm in diameter. Catheter ablation can be effective, but determining a treatment endpoint is difficult and it is undesirable to over-treat. Because determining the treatment endpoint is difficult and because of the undesirability of overtreatment, repeat procedures are common. Accordingly, there remains a need for an improved method of determining a treatment end point.

### SUMMARY

Disclosed is a transducer including: a first optical fiber having an ultrasound generator including a nanoparticulate material on an end thereof; and a second optical fiber having an ultrasound detector on an end thereof, wherein the ultrasound detector includes a Fabry Perot cavity.

Also disclosed is a transducer including: an ultrasound generator including a nanoparticulate material and an ultrasound detector including a Fabry Perot cavity, wherein both the ultrasound generator and the ultrasound detector are disposed on an end of a first optical fiber.

Also disclosed is a system including: the transducer, wherein the first optical fiber and the second optical fiber are disposed together in a same sheath.

Also disclosed is a method of temperature sensing, the method including: providing a transducer; directing an excitation pulse into the first optical fiber to generate an ultrasound signal; sensing an intensity of a reflected interrogation signal from the Fabry Perot cavity to detect the ultrasound signal; and determining a time of flight of the ultrasound signal between a time of the generation and a time of the detection of the ultrasound signal to determine a temperature of a surface.

Also disclosed is a method determining a treatment endpoint, the method including: disposing the transducer over a surface to develop a first thermal image; treating the surface to provide a treated surface; disposing the transducer over the treated surface to develop a second thermal image; comparing the first and the second thermal images to develop a diagnostic image; and determining if the temperature of the treated surface at a depth of 5 mm is 100° C. or greater to determine a treatment endpoint.

Also disclosed is a method of real-time imaging, the method including: disposing the transducer over a surface to develop a thermal image; and displaying the thermal image.

Also disclosed is a method of real-time imaging, the method including: disposing the transducer over a surface to develop a thermal image; while treating the surface to provide a treated surface; and displaying the thermal image.

### BRIEF DESCRIPTION OF THE DRAWINGS

The above and other aspects, advantages and features of this disclosure will become more apparent by describing in further detail exemplary embodiments thereof with reference to the accompanying drawings, in which:

FIG. 1 is a schematic diagram of an embodiment of a fiber optic ultrasound generator;

FIG. 2 is a schematic diagram of an embodiment of a fiber optic ultrasound detector;

FIG. 3 is a schematic diagram of an embodiment of a transducer;

FIG. 4 is a schematic diagram of another embodiment of a transducer;

FIG. 5 is a schematic diagram of yet another embodiment of a transducer;

FIG. 6A is a schematic diagram of an embodiment of a gold nanostructure;

FIG. 6B is a schematic diagram of another embodiment of a gold nanostructure; and

FIG. 7 is a schematic diagram illustrating an embodiment of a method of determining a treatment endpoint.

### DETAILED DESCRIPTION

Using ultrasound to measure temperature is attractive because it is a non-contact method and because it can be combined with ultrasound imaging techniques. Ultrasound temperature measurement is possible because acoustic velocity across a medium is a function of the temperature of the medium. Non-contact temperature measurement would provide a clinical endpoint for ablation procedures. Currently, ablation dosage is estimated based on the time of ablation, which results in repeated procedures because of under ablation. The disclosed transducer is particularly suitable for providing real-time temperature distribution during ablation applications of the ablation location for determination of a clinical endpoint. The disclosed transducer can also be used for ultrasound thermography, in which ultrasound images are used to show temperature changes in a material.

Ultrasonography is described in detail elsewhere, see for example, http://en.wikipedia.org/wiki/Medical_ultrasonography. In short, an acoustic velocity change within a medium, such as tissue, results in a displacement or strain in an ultrasound image. By comparing a first ultrasound image from before a temperature raise to a second ultrasound image collected after the temperature raise, the displacement can be visualized and the temperature of the medium determined. An ultrasound transducer can be scanned across a medium to obtain a two-dimensional amplitude-mode image in which the ultrasound signals are a function of time. Assuming the acoustic velocity is a constant, the time information can represent the depth. However, if the temperature within the tissue is changed, the change in the acoustic velocity will result in a change to the calculated depth, resulting in an additional displacement in the ultrasound image. After performing ultrasound imaging reconstruction processing, a brightness-mode ultrasound image can be obtained, and then by selecting a time window, a cross-section mode image can be obtained at a selected depth. For determination of a clinical endpoint during ablation, temperature at 5 mm depth is desired, and can be determined by comparison of cross-section mode images at 5 millimeters depth before and after ablation.

Disclosed is a fiber optic ultrasound generator and a fiber optic ultrasound detector. The generator uses a nanoparticulate material to generate ultrasound optically. The fiber optic ultrasound detector comprises a Fabry-Perot (FP) interferometer. The ultrasound generator and the detector can be provided on separate optical fibers, or can be integrated onto a single optical fiber so that the generation and detection of the ultrasound can be provided using a single fiber.

Disclosed is a transducer comprising: a first optical fiber having an ultrasound generator comprising a nanoparticulate material on an end thereof; and a second optical fiber having an ultrasound detector on an end thereof, wherein the ultrasound detector comprises a Fabry Perot cavity. Also disclosed is a transducer comprising: an ultrasound generator comprising a nanoparticulate material and an ultrasound detector comprising a Fabry Perot cavity, wherein both the ultrasound generator and the ultrasound detector are disposed on an end of a first optical fiber.

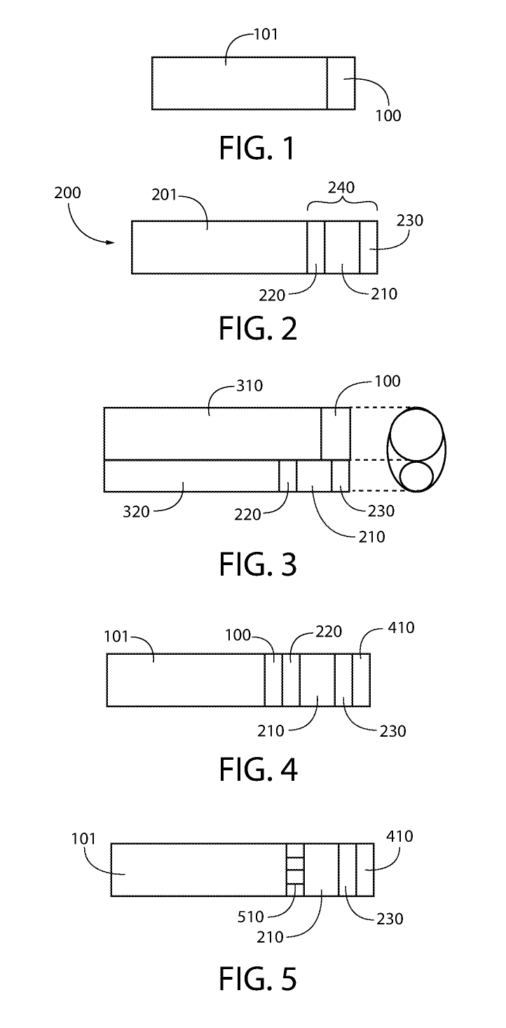

The ultrasound generator 100 may be disposed on an end of an optical fiber 101, as shown schematically in FIG. 1. The ultrasound generator comprises a composite comprising a nanoparticulate material and a polymeric matrix. The nanoparticulate material may comprise gold, aluminum, silver, platinum, copper, carbon black, graphite, graphene, carbon nanotubes, a fullerene, or combination thereof, and may be in the form of nanoparticles, nanorods, nanospheres, or a combination thereof. Gold nanoparticles, gold nanorods, gold-coated carbon nanotubes, gold-coated fullerene, or a combination thereof, are specifically mentioned. The nanoparticulate material may have a particle size of 10 nanometers (nm) to 500 nm, 20 nm to 450 nm, 30 nm to 400 nm, or 40 nm to 350 nm. Also, the nanoparticulate material may have an average particle size of 10 nm to 500 nm, 20 nm to 450 nm, 30 nm to 400 nm, or 40 nm to 350 nm. As used herein, the average particle size can refer to an average longest dimension of the nanoparticulate material. For example, if the nanoparticulate material is a sphere, then the longest dimension would be the diameter, whereas if the nanoparticulate particle is a rod, then the longest dimension would be the length of the rod. In an embodiment, the nanoparticulate material has a relatively narrow size distribution. In an embodiment, 90% of the nanoparticulate material has a particle size within 10%, within 9%, within 8%, or within 7% of the average particle size of the nanoparticulate material. Also, 3 standard deviations of the particle size of the nanoparticulate material may be 1 nm to 50 nm, 2 nm to 45 nm, 3 nm to 40 nm, or 4 nm to 35 nm. Use of gold nanoparticles having an average particle size of 10 nm to 50 nm is specifically mentioned. In another embodiment, use of carbon black is specifically mentioned.

The nanoparticulate material is disposed in the polymeric matrix of the ultrasound generator. The polymeric matrix may comprise an epoxy, polydimethylsiloxane, an ethylene propylene diene terpolymer, a polyacrylate, a polyamide, a polybutadiene, a polycarbonate, a polycarbonate ester, a polyether ketone, a polyether ether ketone, a polyether ketone ketone, a polyethersulfone, a polyester, a polyimide, a polyisoprene, a polyolefin, a polyarylene, a polyphosphazene, a poly(alkyl) (meth)acrylate, a polystyrene, acrylonitrile-butadiene-styrene, styrene-ethylene-butadiene, and methyl methacrylate-butadiene-styrene, polysulfone, polysulfonamide, polyvinyl acetate, polyvinyl chloride, polyvinyl ester, polyvinyl ether, a polyvinyl halide, a polyvinyl nitrile, polyurethane, polyethylene terephthalate, polyethylene naphthalates, a silicone, a copolymer comprising at least one of the foregoing, or combination thereof. The polymeric matrix may have a transparency of 80% to 99%, 82% to 98%, 84% to 97%, or 86% to 96% for light having a wavelength of the interrogation signal, e.g., 1,520 nm to 1,580 nm. Use of poly(p-xylylene), chlorinated poly(p-xylylene), e.g., parylene or parylene-c, or polydimethylsiloxane is specifically mentioned. An embodiment in which the ultrasound generator comprises composite of carbon black nanoparticles in a polymeric matrix comprising polydimethylsiloxane is mentioned.

The ultrasound generator 100, including the nanoparticulate material and the polymeric matrix, are further disclosed in Wu, N., Y. Tian, X. Zou, V. Silva, A. Chery, and X. Wang, “High-efficiency optical ultrasound generation using one-pot synthesized polydimethylsiloxane-gold nanoparticle nanocomposite,” J. Opt. Soc. Am. B, Vol. 29, No. 8, 2016-2020, 2012, the content of which is incorporated herein in its entirety by reference.

The ultrasound detector 200 may also be disposed on an end of an optical fiber 201, as shown in FIG. 2. The ultrasound detector comprises a Fabry Perot cavity 240, and comprises a polymeric layer 210 interposed between first and second parallel reflective layers 220 and 230, respectively. The polymeric layer may comprise an epoxy, polydimethylsiloxane, an ethylene propylene diene terpolymer, a polyacrylate, a polyamide, a polybutadiene, a polycarbonate, a polycarbonate ester, a polyether ketone, a polyether ether ketone, a polyether ketone ketone, a polyethersulfone, a polyester, a polyimide, a polyisoprene, a polyolefin, a polyarylene, a polyphosphazene, a poly(alkyl) (meth)acrylate, a polystyrene, a acrylonitrile-butadiene-styrene copolymer, a styrene-ethylene-butadiene copolymer, a methyl methacrylate-butadiene-styrene copolymer, a polysulfone, a polysulfonamide, polyvinyl acetate, polyvinyl chloride, a polyvinyl ester, a polyvinyl ether, a polyvinyl halide, a polyvinyl nitrile, a polyurethane, polyethylene terephthalate, a polyethylene naphthalate, a polysiloxane, a copolymer comprising at least one of the foregoing, or a combination thereof. Poly(p-xylylene), chlorinated poly(p-xylylene), e.g., parylene or parylene-c, and polydimethylsiloxane are specifically mentioned. The polymeric layer may comprise any suitable material having appropriate durometer, modulus, coating properties on optical fibers, solubility including insolubility in water, and transparency. The polymeric layer may have a durometer of 5 to 70, 6 to 65, 7 to 60, or 8 to 55 on a Shore A scale. The polymeric layer may have a thickness of 5 μm to 100 μm, 10 μm to 80 μm, 15 μm to 70 μm, or 20 μm to 60 μm. The polymeric layer may be substantially insoluble in water, and may have a solubility in water of less than 1% by weight. Also, the polymeric layer may have a transparency of 80% to 99%, 82% to 98%, 84% to 97%, or 86% to 96% for light having a wavelength of the interrogation signal, e.g., 1520 nm to 1580 nm.

The reflective layers of the Fabry Perot cavity may each independently comprise a metal, and may each independently comprise gold, platinum, silver, aluminum, or combination thereof. The reflective layers may each independently have a thickness of 3 nm to 50 nm, 4 nm to 45 nm, 5 nm to 40 nm, or 6 nm to 35 nm. Use of gold reflective layers having a same thickness of 15 nm to 20 nm is specifically mentioned. The reflective layers may each have a transparency of 1% to 20%, 2% to 18%, 3% to 16%, or 4% to 14% for light having a wavelength of an interrogation signal, e.g., an example of the interrogation signal being light having a wavelength of 1520 nm to 1580 nm. In an embodiment reflective layers have a same transparency. The reflective layers each have a reflectivity of 80% to 99%, 82% to 98%, 84% to 97%, or 86% to 96% for light having a wavelength of an interrogation signal, e.g., an example being light having a wavelength of 1,520 nm to 1,580 nm. In an embodiment reflective layers have a same reflectivity.

The ultrasound detector 200 may be provided by depositing, e.g., by sputtering such as direct-current sputtering, a first reflective layer on the end of an optical fiber, disposing the polymeric layer on the first reflective layer by, for example, vacuum deposition, and then disposing the second reflective layer on the polymeric layer. If desired, an additional layer of the protective layer may be disposed on the second reflective layer to provide a protective layer. The protective layer may have a thickness of 0.5 μm to 5 μm.

The first optical fiber, and the second optical fiber, if present, may each independently be a single mode fiber or a multimode fiber. An embodiment in which the first optical fiber is a multimode fiber, and the second optical fiber is a single mode fiber is mentioned. In another embodiment, the first optical fiber is a single mode fiber, and the second optical fiber is a multimode fiber. The first optical fiber and the second optical fiber, if present, may each independently have any suitable diameter, e.g., a diameter of 80 micrometers (m) to 1000 μm, 90 μm to 900 μm, or 100 μm to 800 μm.

Each fiber individually can comprise a fiber coating. The coated first optical fiber and the coated second optical fiber may each independently have any suitable diameter, e.g., a diameter of 80 μm to 1000 μm, 90 μm to 900 μm, or 100 μm to 800 μm. In an embodiment, a diameter of the coated first optical fiber may be greater than the diameter of the coated second optical fiber, for example, by greater than or equal to 50%. The diameter of the coated first optical fiber may be 500 to 1000 μm, or 650 to 800 μm. The diameter of the coated second optical fiber may be 100 to 500 μm, or 150 to 300 μm.

The first optical fiber and the second optical fiber may comprise an uncoated length adjacent to the tip of the respective fibers. The uncoated length can be 2 to 50 mm, or 5 to 20 mm, or 5 to 15 mm. When disposed in a fiber bundle, the uncoated length of the first optical fiber and the uncoated length of the second optical fiber may have major axes that are parallel to each other or within 5° of each other. The diameter of the uncoated first optical fiber can be 200 to 600 μm, or 300 to 500 μm, or 400 to 500 μm. The diameter of the uncoated second optical fiber may be 50 to 300 μm, or 75 to 200 μm, or 100 to 150 μm.

In an embodiment, the ultrasound generator 100 may be disposed on a first optical fiber 310, and the ultrasound detector 200 may be disposed on a second fiber 320 to form a fiber bundle. An example of a fiber bundle is shown in FIG. 3, where the left-hand image is a side view and the right-hand image is an end view. The fiber bundle can comprise the ultrasound generator disposed on a first optical fiber and the ultrasound detector disposed on a second optical fiber. The fiber bundle can comprise a bundle coating that can bundle the first optical fiber and the second optical fiber together. The fiber bundle can be flexible. A steerable catheter can comprise the fiber bundle.

The fiber bundle can be prepared by one or both of coating or adhering a first optical fiber comprising an ultrasound generator and a second optical fiber comprising an ultrasound detector to form the fiber bundle. The first optical fiber can be prepared by removing a portion of a coating from the first optical fiber to form a first tip portion and disposing the ultrasound generator on a generator end of the first tip portion. The second optical fiber can be prepared by removing a portion of a coating from the second optical fiber to form a second tip portion and disposing the ultrasound detector on a detector end of the second tip portion. The fiber bundle can be flexible.

In another embodiment, as shown in FIG. 4, ultrasound detector may be disposed between the optical fiber 101 and the ultrasound generator 100. The ultrasound generator may be directly on the Fabry Perot cavity 210 of the ultrasound detector or may be directly on reflective layer 220. The reflective layers and the polymeric layer of the Fabry Perot cavity may be provided by coating, for example. Also, a protective layer 410 may be provided to protect the device. The protective layer may comprise any suitable polymer, such as the polymer disclosed for the polymeric layer.

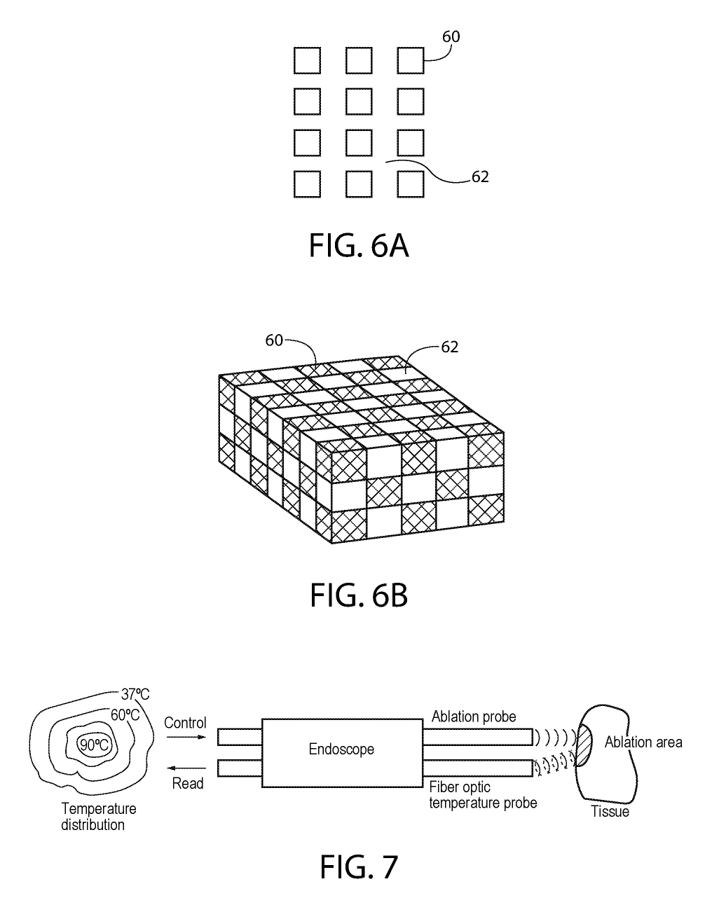

In yet another embodiment, a nanostructure may be used both as the ultrasound generator and as the first reflective layer 510, which is disposed directly on the optical fiber 101, as shown in FIG. 5. The nanostructure may comprise a matrix or checkerboard structure, as shown in FIG. 6A and FIG. 6B, wherein a distance between adjacent features can be 5 nm to 100 nm, 10 nm to 75 nm, or 15 nm to 50 nm, and a size, for example, a length and/or width of each feature can each independently be 5 nm to 100 nm, 10 nm to 75 nm, or 15 nm to 50 nm. The features may comprise a metal such as gold, for example. The checkerboard-like nanostructure is understood to enhance photoacoustic generation efficiency. In an embodiment, a nanostructure includes a combination of gold cells 60 and polymer cells 62, such as polymer cells of polydimethylsiloxane (PDMS). In an embodiment, a polysiloxane layer such as a PDMS layer is deposited on a surface of a multimodal fiber. In an embodiment, a mold is employed to fabricate a pattern of gold cells on a surface. The gold layer is then deposited on top of the PDMS layer. The deposition of gold and PDMS is performed alternately. The dimension of the gold cells can be controlled to a range of 10 nanometers to 100 nanometers. Because different sizes of gold cells correspond to different maximum absorption wavelengths, a phased array technique, such as is known in the art, can be employed. One aspect of the invention includes exciting different wavelengths of laser onto different dimensions of gold cells.

In another embodiment, the gold nanostructure shown in FIG. 6B can be fabricated by a technique wherein the sequence of the deposition of gold and the polysiloxane such as PDMS is reversed. Generally speaking, the gold layer is deposited on a surface of the multimodal fiber first, and a focused ion beam is introduced to generate a pattern. Thereafter, a polysiloxane layer such as a PDMS layer is coated on the gold pattern. On the PDMS layer the focused ion beam is introduced again to fabricate the PDMS pattern. The focused ion beam etching and the deposition are performed alternately. The dimensions of the gold cell and the PDMS cell can be controlled very precisely by the focused ion beam.

As noted above, the transducer is useful for determining the temperature of the surface. Disclosed is a system comprising: the transducer, wherein the first optical fiber and the second optical fiber are disposed together in a same sheath. Also disclosed is a system comprising: the transducer; and a first laser configured to provide an excitation pulse for generating ultrasound, and a second laser configured to provide an interrogation signal to determine a distance between the reflective layers of the Fabry Perot cavity.

An ultrasound signal may be generated when the nanoparticulate material is contacted by the excitation pulse from the first laser. The wavelength of the excitation pulse may be any suitable wavelength, and may be 400 nm to 1,200 nm, 450 nm to 1,100 nm, or 500 nm to 1,000 nm. Use of a 527 nm excitation pulse in combination with 20 nm gold nanoparticles, or a 1,064 nm excitation pulse in combination with carbon black nanoparticles are mentioned. The pulse width can be any suitable width, and can be 50 nanoseconds (ns) to 200 ns, or 75 ns to 150 ns. In an embodiment, the excitation pulse has an optical energy of 11 microjoules per pulse, and results in an ultrasound signal having a pressure of 780 kilopascals at 1.2 millimeters. When contacted by the excitation pulse, the nanoparticulate material absorbs the excitation pulse, causing it to heat and thermally expand and contract upon cooling, which results in the generation of sound. The frequency of the ultrasound can be selected by the frequency of the excitation pulses from the first laser and the thickness of the polymeric matrix. A thinner polymeric matrix provides a higher frequency and a broader bandwidth. A shorter pulse width of the laser source will provide to a broader bandwidth. Broader bandwidth can generate clearer images while higher generation efficiency can generate stronger ultrasound, and therefore, increased dynamic range. However, a thinner polymeric matrix will result in reduced sound generation efficiency. Therefore a trade-off exists between the bandwidth and the generation efficiency. Also, the intensity of the ultrasound signal can be selected by the laser power. The intensity of the ultrasound signal increases with increasing laser power. A thickness of the polymeric matrix may be 5 μm to 100 μm, 10 μm to 75 μm, 15 μm to 50 μm, or 20 μm to 25 μm. Use of a 30 μm thick polymeric matrix to provide ultrasound having a frequency of 20 megahertz (MHz) is mentioned. The laser fluence may be 1 millijoule per square centimeter per pulse (mJ/cm²/pulse) to 100 mJ/cm²/pulse, 2 mJ/cm²/pulse to 75 mJ/cm²/pulse, or 3 mJ/cm²/pulse to 50 mJ/cm²/pulse. Use of 10 mJ/cm²/pulse is mentioned. The composite comprising the nanoparticulate material and the polymeric matrix may be disposed on the end of the optical fiber by any suitable method, such as coating, for example.

Also, the direction of the ultrasound may be focused by selection of a shape of the ultrasound generator, specifically a shape of the polymeric matrix. Use of a flat shape to provide a diverse ultrasound signal, or a dome-shaped, elliptically-shaped, or a parabolically-shaped ultrasound generator to provide a focused ultrasound signal is mentioned.

To interrogate the Fabry Perot cavity, a second laser may be used to provide an interrogation signal which generates a reflection interference spectrum. The wavelength of the interrogation signal may be any suitable wavelength, and may be 600 nm to 1,800 nm, 700 nm to 1,700 nm, or 800 nm to 1,600 nm. Use of an interrogation signal having a wavelength of 1520 nm to 1560 nm is mentioned. Use of a continuous laser interrogation signal is preferred, however a pulse can be used. When ultrasound signals contact the polymeric layer, the polymeric layer is deformed, causing the cavity length of the Fabry Perot cavity to change, resulting in a shift in the reflection interference spectrum. By measuring the shift of the interference spectrum, the ultrasound signal can be demodulated. Additional detail is provided in U.S. Patent Publication No. 2013/0319123, the content of which is incorporated herein by reference in its entirety.

The transducer is useful for determining the temperature of the surface. Furthermore, because the transducer can be housed within a sheath of an endoscope, and because it provides a non-contact method of determining temperature, it is practically suitable for providing a clinical endpoint during ablation. During an ablation procedure, a temperature distribution of a treated surface, e.g., an ablated area, can be determined using the transducer.

Disclosed is a method of temperature sensing, the method comprising: providing a transducer; directing an excitation pulse into the first optical fiber to generate an ultrasound signal; sensing an intensity of a reflected interrogation signal from the Fabry Perot cavity to detect the ultrasound signal; and determining a time of flight of the ultrasound signal between a time of the generation and a time of the detection of the ultrasound signal to determine a temperature of a surface.

FIG. 7 illustrates an embodiment of a method of determining a treatment endpoint. The method comprises: disposing the transducer over a surface to develop a first thermal image; treating the surface to provide a treated surface; disposing the transducer over the treated surface to develop a second thermal image; comparing the first and the second thermal images to develop a diagnostic image; and determining if the temperature of the treated surface at a depth of 5 mm is 100° C. or greater to determine a treatment endpoint.

Also disclosed is a method of imaging, the method comprising: disposing the transducer over a surface to develop a thermal image; while treating the surface to provide a treated surface; and displaying the thermal image. The imaging may be provided in real time, thereby providing feedback on the progress of an ablation and more accurate treatment.

The treated surface may be any suitable surface and may be human tissue, for example.

Alternatively, the treatment may be omitted if desired. Disclosed is a method of imaging, the method comprising: disposing the transducer over a surface to develop a thermal image and displaying the thermal image.

Set forth below are embodiments of the transducer, methods of making, methods of using, and uses thereof.

### Embodiment 1

A transducer comprising: a first optical fiber having an ultrasound generator comprising a nanoparticulate material on an end thereof; and a second optical fiber having an ultrasound detector on an end thereof, wherein the ultrasound detector comprises a Fabry Perot cavity.

### Embodiment 2

A transducer comprising: an ultrasound generator comprising a nanoparticulate material and an ultrasound detector comprising a Fabry Perot cavity, wherein both the ultrasound generator and the ultrasound detector are disposed on an end of a first optical fiber.

### Embodiment 3

The transducer of any of embodiments 1 to 2, wherein the ultrasound generator comprises a composite comprising a nanoparticulate material and a polymeric matrix.

### Embodiment 4

The transducer of any of embodiments 1 to 3, wherein the nanoparticulate material has a particle size of 10 nm to 500 nm.

### Embodiment 5

The transducer of any of embodiments 1 to 4, wherein the nanoparticulate material has an average particle size of 10 nm to 500 nm.

### Embodiment 6

The transducer of any of embodiments 1 to 5, wherein 90% of the nanoparticulate material has a particle size within 10% of the average particle size of the nanoparticulate material.

### Embodiment 7

The transducer of any of embodiments 1 to 6, wherein 3 standard deviations of the particle size of the nanoparticulate material is 1 nm to 50 nm.

### Embodiment 8

The transducer of any of embodiments 1 to 7, wherein the nanoparticulate material comprises gold, aluminum, silver, platinum, copper, carbon black, graphite, graphene, carbon nanotubes, a fullerene, or combination thereof.

### Embodiment 9

The transducer of any of embodiments 1 to 8, wherein the nanoparticulate material is in a form of nanoparticles, nanorods, nanospheres, or combination thereof.

### Embodiment 10

The transducer of any of embodiments 1 to 9, wherein the nanoparticulate material comprises gold nanoparticles, gold nanorods, gold coated carbon nanotubes, gold coated fullerene, or a combination thereof.

### Embodiment 11

The transducer of any of embodiments 1 to 10, wherein the nanoparticulate material comprises gold nanoparticles having a particle size of 10 nanometers to 500 nanometers.

### Embodiment 12

The transducer of any of embodiments 1 to 11, wherein the nanoparticulate material is disposed in a polymeric matrix.

### Embodiment 13

The transducer of any of embodiments 1 to 12, wherein the nanoparticulate material is disposed in a polymeric matrix; and wherein the polymeric matrix comprises an epoxy, polydimethylsiloxane, an ethylene propylene diene terpolymer, a polyacrylate, a polyamide, a polybutadiene, a polycarbonate, a polycarbonate ester, a polyether ketone, a polyether ether ketone, a polyether ketone ketone, a polyethersulfone, a polyester, a polyimide, a polyisoprene, a polyolefin, a polyarylene, a polyphosphazene, a poly(alkyl) (meth)acrylate, a polystyrene, acrylonitrile-butadiene-styrene, styrene-ethylene-butadiene, and methyl methacrylate-butadiene-styrene, polysulfone, polysulfonamide, polyvinyl acetate, polyvinyl chloride, polyvinyl ester, polyvinyl ether, a polyvinyl halide, a polyvinyl nitrile, polyurethane, polyethylene terephthalate, polyethylene naphthalates, a silicone, a copolymer comprising at least one of the foregoing, or combination thereof, each of which may be substituted or unsubstituted with one more of a fluoro, chloro, bromo, iodo, and astatino substituent.

### Embodiment 14

The transducer of any of embodiments 1 to 13, wherein the nanoparticulate material is disposed in a polymeric matrix; and wherein the polymeric matrix comprises poly(p-xylylene), a chlorinated poly(p-xylylene), or polydimethylsiloxane.

### Embodiment 15

The transducer of any of embodiments 1 to 14, wherein the nanoparticulate material is disposed in a polymeric matrix; and wherein the polymeric matrix has a transparency of 80% to 99% for light having a wavelength of 1520 nm to 1580 nm.

### Embodiment 16

The transducer of any of embodiments 1 to 15, wherein the Fabry Perot cavity comprises a polymeric layer interposed between parallel reflective layers.

### Embodiment 17

The transducer of any of embodiments 1 to 16, wherein the Fabry Perot cavity comprises a polymeric layer interposed between parallel reflective layers; and wherein the polymeric layer comprises an epoxy, polydimethylsiloxane, poly(p-xylylene) a chlorinated poly(p-xylylene), parylene, parylene-c, an ethylene propylene diene terpolymer, a polyacrylate, a polyamide, a polybutadiene, a polycarbonate, a polycarbonate ester, a polyether ketone, a polyether ether ketone, a polyether ketone ketone, a polyethersulfone, a polyester, a polyimide, a polyisoprene, a polyolefin, a polyarylene, a polyphosphazene, a poly(alkyl) (meth)acrylate, a polystyrene, acrylonitrile-butadiene-styrene, styrene-ethylene-butadiene, and methyl methacrylate-butadiene-styrene, polysulfone, polysulfonamide, polyvinyl acetate, polyvinyl chloride, polyvinyl ester, polyvinyl ether, a polyvinyl halide, a polyvinyl nitrile, polyurethane, polyethylene terephthalate, polyethylene naphthalates, a silicone, a copolymer comprising at least one of the foregoing, or combination thereof, each of which may be substituted or unsubstituted with one more of a fluoro, chloro, bromo, iodo, and astatino substituent.

### Embodiment 18

The transducer of any of embodiments 1 to 17, wherein the Fabry Perot cavity comprises a polymeric layer interposed between parallel reflective layers; and wherein the reflective layers each independently comprise a metal.

### Embodiment 19

The transducer of any of embodiments 1 to 18, wherein the Fabry Perot cavity comprises a polymeric layer interposed between parallel reflective layers; and wherein the reflective layers each independently comprise gold, platinum, silver, aluminum, or combination thereof.

### Embodiment 20

The transducer of any of embodiments 1 to 19, wherein the Fabry Perot cavity comprises a polymeric layer interposed between parallel reflective layers; and wherein the reflective layers each independently have a thickness of 3 nm to 50 nm.

### Embodiment 21

The transducer of any of embodiments 1 to 20, wherein the Fabry Perot cavity comprises a polymeric layer interposed between parallel reflective layers; and wherein the reflective layers each consist of gold.

### Embodiment 22

The transducer of any of embodiments 1 to 21, wherein the Fabry Perot cavity comprises a polymeric layer interposed between parallel reflective layers; and wherein the reflective layers have a same thickness.

### Embodiment 23

The transducer of any of embodiments 1 to 22, wherein the Fabry Perot cavity comprises a polymeric layer interposed between parallel reflective layers; and wherein the reflective layers each have a transparency of 1% to 20% for light having a wavelength of an interrogation signal.

### Embodiment 24

The transducer of any of embodiments 1 to 23, wherein the Fabry Perot cavity comprises a polymeric layer interposed between parallel reflective layers; and wherein the polymeric layer has a transparency of at least 80% for light having a wavelength of the interrogation signal.

### Embodiment 25

The transducer of any of embodiments 1 to 24, wherein the Fabry Perot cavity comprises a polymeric layer interposed between parallel reflective layers; and wherein the reflective layers have a same transparency.

### Embodiment 26

The transducer of any of embodiments 1 to 25, wherein the Fabry Perot cavity comprises a polymeric layer interposed between parallel reflective layers; and wherein the reflective layers each have a reflectivity of 80% to 99%.

### Embodiment 27

The transducer of any of embodiments 1 to 26, wherein the Fabry Perot cavity comprises a polymeric layer interposed between parallel reflective layers; and wherein the reflective layers have a same reflectivity.

### Embodiment 28

The transducer of any of embodiments 1 to 27, wherein the Fabry Perot cavity comprises a polymeric layer interposed between parallel reflective layers; and wherein the polymeric layer has a durometer of 5 to 70 on a Shore A scale.

### Embodiment 29

The transducer of any of embodiments 1 to 28, wherein the Fabry Perot cavity comprises a polymeric layer interposed between parallel reflective layers; and wherein the polymeric layer has a thickness of 20 μm to 50 μm.

### Embodiment 30

The transducer of any of embodiments 1 to 29, wherein the first optical fiber and the second optical fiber, if present, each independently have a diameter of 80 μm to 1000 μm.

### Embodiment 31

The transducer of any of embodiments 1 to 30, wherein the first optical fiber and the second optical fiber, if present, are each independently a single mode fiber or a multi-mode fiber.

### Embodiment 32

The transducer of any of embodiments 1 to 31, wherein at least one of the first optical fiber and the second optical fiber, if present, is a single mode fiber.

### Embodiment 33

The transducer of any of embodiments 2 to 32, wherein the ultrasound generator is directly on the Fabry Perot cavity of the ultrasound detector.

### Embodiment 34

The transducer of any of embodiments 2 to 33, wherein the ultrasound generator is between the optical fiber and the Fabry Perot cavity of the ultrasound detector.

### Embodiment 35

The transducer of any of embodiments 1 to 34, wherein the Fabry Perot cavity comprises a polymeric layer interposed between parallel reflective layers; and further comprising a first laser configured to provide an excitation pulse for generating ultrasound and a second laser configured to provide an interrogation signal to determine a distance between the reflective layers of the Fabry Perot cavity.

### Embodiment 36

The transducer of any of embodiments 1 to 35, wherein the transducer is a temperature sensor.

### Embodiment 37

A system comprising: the transducer of any of embodiments 1 and 3 to 36, wherein the first optical fiber and the second optical fiber are disposed together in a same sheath.

### Embodiment 38

A system comprising: the transducer of any of embodiments 1 to 36; wherein the Fabry Perot cavity comprises a polymeric layer interposed between parallel reflective layers; and a first laser configured to provide an excitation pulse for generating ultrasound, and a second laser configured to provide an interrogation signal to determine a distance between the reflective layers of the Fabry Perot cavity.

### Embodiment 39

A method of temperature sensing, the method comprising: providing a transducer according to any of embodiments 1 to 36; directing an excitation pulse into the first optical fiber to generate an ultrasound signal; sensing an intensity of a reflected interrogation signal from the Fabry Perot cavity to detect the ultrasound signal; and determining a time of flight of the ultrasound signal between a time of the generation and a time of the detection of the ultrasound signal to determine a temperature of a surface.

### Embodiment 40

A method determining a treatment endpoint, the method comprising: disposing the transducer of any of embodiments 1 to 36 over a surface to develop a first thermal image; treating the surface to provide a treated surface; disposing the transducer over the treated surface to develop a second thermal image; comparing the first and the second thermal images to develop a diagnostic image; and determining if the temperature of the treated surface to determine a treatment endpoint.

### Embodiment 41

The method of determining treatment end point of embodiment 39, wherein the treating of the surface is ablation, and wherein the treatment endpoint is determined by determining if the temperature of the treated surface at a depth of 5 mm is 100° C. or greater.

### Embodiment 42

A method of real-time imaging, the method comprising: disposing the transducer of any of embodiments 1 to 36 over a surface to develop a thermal image; and displaying the thermal image.

### Embodiment 43

A method of real-time imaging, the method comprising: disposing the transducer of any of embodiments 1 to 36 over a surface to develop a thermal image; while treating the surface to provide a treated surface; and displaying the thermal image.

### Embodiment 44

The method of embodiment 42, wherein the method is a method of determining an end point of a treatment.

### Embodiment 45

The method of any of embodiments 38 to 42, wherein the method is a method of real-time imaging.

### Embodiment 46

The method of any of embodiments 35 to 42, wherein the surface is a biological tissue.

### Embodiment 47

A steerable catheter comprising the transducer of any one of the preceding embodiments.

The disclosure now has been described with reference to the accompanying drawings, in which various embodiments are shown. This disclosure may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third,” etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

All references cited herein are incorporated by reference in their entirety.

1. A transducer comprising:
a first optical fiber having an ultrasound generator comprising a nanoparticulate material on an end thereof; and
an ultrasound detector comprising a Fabry Perot cavity;
wherein the ultrasound detector is located on an end of a second optical fiber or on the end of the first optical fiber.

2. The transducer of claim 1, wherein the ultrasound generator comprises a composite comprising the nanoparticulate material and a polymeric matrix.

3. The transducer of claim 2, wherein the polymeric matrix comprises poly(p-xylylene), a chlorinated poly(p-xylylene), or polydimethylsiloxane.

4. The transducer of claim 1, wherein the nanoparticulate material has an average particle size of 10 nm to 500 nm.

5. The transducer of claim 1, wherein the nanoparticulate material comprises gold, aluminum, silver, platinum, copper, carbon black, graphite, graphene, carbon nanotubes, a fullerene, or a combination thereof.

6. The transducer of claim 1, wherein the nanoparticulate material comprises gold nanoparticles, gold nanorods, gold coated carbon nanotubes, gold coated fullerene, or a combination thereof.

7. The transducer of claim 1, wherein the Fabry Perot cavity comprises a polymeric layer interposed between parallel reflective layers.

8. The transducer of claim 7, wherein the reflective layers each independently comprise gold, platinum, silver, aluminum, or a combination thereof.

9. The transducer of claim 7, wherein the reflective layers each independently have a thickness of 3 nm to 50 nm.

10. The transducer of claim 7, wherein the polymeric layer has a thickness of 20 μm to 50 μm.

11. The transducer of claim 7, further comprising a first laser configured to provide an excitation pulse for generating ultrasound and a second laser configured to provide an interrogation signal to determine a distance between the reflective layers of the Fabry Perot cavity.

12. The transducer of claim 1, wherein the first optical fiber and the second optical fiber, if present, each independently have a diameter of 80 μm to 1,000 μm.

13. The transducer of claim 1, wherein the ultrasound detector is located on the end of the first optical fiber; and wherein the ultrasound generator is directly on the Fabry Perot cavity of the ultrasound detector.

14. The transducer of claim 1, wherein the ultrasound detector is located on the end of the first optical fiber; and wherein the ultrasound generator is between the optical fiber and the Fabry Perot cavity of the ultrasound detector.

15. The transducer of claim 1, wherein the transducer is a temperature sensor.

16. The transducer of claim 1, wherein the ultrasound detector is located on the end of the second optical fiber; and wherein the first optical fiber and the second optical fiber are optionally disposed together in a same sheath.

17. A transducer comprising:
an optical fiber having an ultrasound generator and an ultrasound detector disposed on an end of the optical fiber;
wherein the ultrasound generator comprises a nanoparticulate material and the ultrasound detector comprises a Fabry Perot cavity;
wherein the ultrasound detector is located in between the end of the optical fiber and the ultrasound generator.

18. A system comprising:
the transducer of claim 1; wherein the Fabry Perot cavity comprises a polymeric layer interposed between parallel reflective layers; and
a first laser configured to provide an excitation pulse for generating ultrasound, and
a second laser configured to provide an interrogation signal to determine a distance between the reflective layers of the Fabry Perot cavity.

19. A method of temperature sensing, the method comprising:
providing a transducer according to claim 1;
directing an excitation pulse into the first optical fiber to generate an ultrasound signal;
sensing an intensity of a reflected interrogation signal from the Fabry Perot cavity to detect the ultrasound signal; and
determining a time of flight of the ultrasound signal between a time of the generation and a time of the detection of the ultrasound signal to determine a temperature of a surface.

20. A method determining a treatment endpoint, the method comprising:
disposing the transducer of claim 1 over a surface to develop a first thermal image;
treating the surface to provide a treated surface;
disposing the transducer over the treated surface to develop a second thermal image;
comparing the first and the second thermal images to develop a diagnostic image; and
determining if the temperature of the treated surface to determine a treatment endpoint.
